(19) United States
(12) Patent Application Publication
(10) Pub. No.: US 2016/0346171 A1
(43) Pub. Date: Dec. 1, 2016

(54) ORAL COMPOSITION AND CONFECTION

(71) Applicants: Kazuhisa Maeda, Osaka (JP); TEIKYO UNIVERSITY, Tokyo (JP); UHA MIKAKUTO CO., LTD., Osaka (JP)

(72) Inventors: Shigeru Abe, Tokyo (JP); Kazumi Hayama, Tokyo (JP); Miki Takahashi, Tokyo (JP); Motofumi Suzuki, Tokyo (JP); Akinobu Kishi, Yamatokoriyama-shi (JP); Nobuya Sato, Yamatokoriyama-shi (JP); Taiji Matsukawa, Yamatokoriyama-shi (JP); Yasumasa Yamada, Yamatokoriyama-shi (JP); Ichiro Yamada, Yamatokoriyama-shi (JP); Kazuhisa Maeda, Osaka (JP)

(21) Appl. No.: 14/724,746

(22) Filed: May 28, 2015

(51) Int. Cl. A61K 8/02; A61K 8/49; A61K 8/34; A23G 4/12; A61K 8/36; A61K 8/33; A23G 3/36; A61Q 11/00; A61K 8/97

(52) U.S. Cl. CPC A61K 8/0204; A61Q 11/00; A61K 8/498; A61K 8/34; A61K 8/97; A61K 8/361; A61K 8/33; A23G 3/364; A23G 4/12; A61K 2800/92; A61K 2800/592; A23V 2002/00

(57) ABSTRACT

There are provided an oral composition and a confection that exhibit activity equivalent to or higher than that of a decanoic acid-containing composition, and a method for producing such an oral composition. The oral composition and the confection contain at least three ingredients: cinnamon, low molecular proanthocyanidin, and menthol. The method for producing, the oral composition includes the steps of: dissolving at least 0.2 to 1.0% by weight of low molecular proanthocyanidin in 0.2 to 2.0% by weight of at least one water-soluble solvent selected from glycerin or propylene glycol to form a solution; kneading at least 0.1 to 0.5% by weight of cinnamon and 0.1 to 0.4% by weight of menthol into sugar-free base dough to prepare candy- or gel-like kneaded dough; and mixing the solution with the kneaded dough.

### BACKGROUND OF THE INVENTION

(1) Field of the Invention

The present invention relates to an oral composition and a confection having the effect of improving oral hygiene.

(2) Description of Related Art

In a healthy condition, the oral cavity and the mucous membrane surface are usually kept moist, and mucous membrane-forming epithelial cells and glandular cells secreting saliva and so on protect the environment by producing various complex carbohydrates and proteins. In the oral cavity, various microorganisms also live as normal flora usually without being pathogenic. However, in elderlies, people with reduced immune strength, or patients with drug therapy-induced superinfection or opportunistic infection, microorganisms can proliferate on the oral mucosa even though they are usually less pathogenic, so that they can cause persistent symptoms to produce long-term pain or discomfort, which can reduce the quality of life.

Oral candidiasis is a cause of such deterioration of oral hygiene. Abe S. et al. Medical Mycology Research. 2011;2(1):15-20 discloses that various plant-derived substances are effective in treating candidiasis and examples of such substances include low molecular proanthocyanidin (trade name; Oligonol (registered trademark), AMINO UP CHEMICAL Co., Ltd.), cinnamon containing cinnamaldehyde as a main component, decanoic acid (capric acid), which is a linear fatty acid of 10 carbon atoms, eugenol, etc.

Hayama K. et al. Jpn S Med Mycol. 2010;51;137-142 discloses that at least 313 μg/ml, of Oligonol mentioned above inhibits in vitro Candida mycelial growth (80% inhibition) and that in an oral candidiasis mouse model, a 20 mg/ml, Oligonol solution ameliorated the tongue symptoms when administered three times (50 μL at a time) into the oral cavity. It should be noted that Oligonol has the disadvantage that when its concentration is as high as 20 mg/mL, it has strong astringency and can significantly deteriorate taste.

Taguchi Y, et al. Med Mycol J. 2011;52:1145-152 discloses that in an oral candidiasis mouse model, a 19.5 mg/mL cinnamaldehyde solution ameliorated the tongue symptoms when administered three times (50 μL at a time) into the oral cavity.

Inoue S. et al, Med Mycol J. 2012;53:33-40 discloses that at least 12.5 μg/mL or 3.13 μg/mL of decanoic acid mentioned above inhibits in vitro Candida mycelial growth by 90% or 50%.

As is apparent from these documents, decanoic acid has been considered to be promising among various plant-derived substances because a small amount of it has anti-Candida activity.

In addition, to the inventors knowledge, there has been no known oral composition or confection for ameliorating deterioration of oral hygiene, for example, no known oral composition or confection formulated by focusing on anti-Candida activity.

JP 2013-40156 A also shows the superiority of decanoic acid and discloses that at least 3.13 μg/mL of decanoic acid, at least 25 μg/mL of geraniol at least 200 μg/mL of eugenol, and at least 50 μg/mL of citral each independently inhibited in vitro Candida mycelial growth by 95% and that a high synergistic effect was observed when decanoic acid was used in combination with either geraniol, eugenol, or citral. JP 2013-40156 A also discloses that in an oral candidiasis mouse model, a 1% (=10 mg/mL) decanoic acid solution, a 3% ginger essential oil solution, or a combination of 1% decanoic acid and a 3% ginger essential oil-containing solution ameliorated the tongue symptoms when administered three times (50 μL at a time in all cases) into the oral cavity and that the combination of decanoic acid and ginger essential oil was most effective. The ginger essential oil has a citral content of 24.9% and a geraniol content of 4.7%.

These documents report the results achieved by the inventors and collaborators, except that Abe S, et al. Medical Mycology Research. 2011;2(1):15-20 is a review article containing the results achieved by the inventors. The inventors have made diligent studies on anti-Candida activity of a large number of plant-derived substances including those disclosed in Abe S. et al. Medical Mycology Research. 2011;2(1)15-20 while focusing on decanoic acid, which is active even in a small amount. As a result, the inventors have been assured of the superiority of decanoic acid based on the finding that decanoic acid is more effective when used in combination with other plant-derived substances (e.g., citral) as shown in JP 2013-40156 A.

Under these circumstances, the inventors have made an evaluation of decanoic acid using an oral candidiasis mouse model because the superiority of decanoic acid has been demonstrated mainly in vitro tests. In the evaluation, the inventors have found that although not containing decanoic acid, some compositions are as effective in improving oral hygiene as decanoic acid-containing compositions.

### SUMMARY OF THE INVENTION

### Problems to Be Solved by the Invention

Specifically, an object of the present invention is to provide an oral composition that exhibits activity equivalent to or higher than that of a decanoic acid-containing composition and in particular to provide such an oral composition in the form of an oral composition and a confection. To date, there has been no known oral composition or confection designed to improve oral hygiene. Thus, another object of the present invention is to provide an oral composition and a confection having the effect of improving oral hygiene and to provide a method for producing such an oral composition.

### Means for Solving the Problems

The problems can be solved by the present invention described as follows:
[1] An oral composition including at least three ingredients: cinnamon; low molecular proanthocyanidin; and menthol.
[2] The oral composition according to [1], further including: at least one ingredient selected from a middle chain fatty acid, a middle chain fatty acid ester, and citral.
[3] The oral composition according to [1], further including: a thickener.
[4] The oral composition according to [1], wherein the cinnamon is a composition containing cinnamaldehyde.
[5] The oral composition according to [1], further including at least one ingredient selected from decanoic acid or ethyl decanoate as a middle chain fatty acid or a middle chain fatty acid ester.

[6] The oral composition according to [1], wherein the low molecular proanthocyanidin includes Oligonol (trademark).

[7] The oral composition according to any one of [1] to [6], which contains at least 0.28 mg or more of cinnamon, 0.5 mg or more of low molecular proanthocyanidin, and 0.2 mg or more of menthol per oral dosage unit.

[8] The oral composition according to [2], which contains at least one selected from 0.008 mg or more of a middle chain fatty acid, 0.008 mg or more of a middle chain fatty acid ester, or 0.14 mg more of citral per oral dosage unit.

[9] The oral composition according to [7], which promotes secretion of salivary adiponectin when orally taken to an organism.

[10] A confection including at least three ingredients: cinnamon: low molecular proanthocyanidin; and menthol.

[11] The confection according to [10], further including: at least one ingredient selected from a middle chain fatty acid, a middle chain fatty acid ester, and citral.

[12] The confection according, to [10] further including: a thickener.

[13] The confection according to [10] , wherein the cinnamon is a composition containing cinnamaldehyde.

[14] The confection according to [10] further including at least one ingredient selected from decanoic acid or ethyl decanoate as a middle chain fatty acid or a middle chain fatty acid ester.

[15] The confection according to [10], wherein the low molecular proanthocyanidin includes Oligonol (trademark).

[16] The confection according to any one of [10] to [15], which can be dissolved in a mouth when taken and contains at least 0.28 mg or more of cinnamon, 0.5 mg or more of low molecular proanthocyanidin, and 0.2 mg or more of menthol per orally dissolving unit.

[17] The confection according to [11] which can be dissolved in the mouth when taken and contains at least one selected from 0.008 mg or more of a middle chain fatty acid, 0.008 mg or more of a middle chain fatty acid ester, or 0.14 mg or more of citral per orally dissolving unit.

[18] The confection according to [16], which is in the form of a candy product or a gel product and has the effect of improving oral hygiene when dissolved in the mouth of an organism.

[19] The confection according to [16], which is in the form of a candy product or a gel product and has the effect of promoting secretion of salivary adiponectin when dissolved in the mouth of an organism.

[20] A method for producing an oral composition, the method including the steps of:

dissolving at least 0.2 to 1.0% by weight of low molecular proanthocyanidin in 0.2 to 2.0% by weight of at least one water-soluble solvent selected from glycerin or propylene glycol to form a solution;

kneading at least 0.1 to 0.5% by weight of cinnamon and 0.1 to 0.4% by weight of menthol into sugar-free, candy- or gel-like, base dough to prepare candy- or gel-like kneaded dough; and

mixing the solution with the kneaded dough.

The present invention relates to an oral composition and a confection. Among them, the confection may be regarded as a specific dosage form of the oral composition. Hereinafter, the oral composition, which is an embodiment of the present invention with no limitation on dosage form will be mainly described.

As used herein, the term “the effect of improving oral hygiene” is intended to include, but not be limited to, the effect of inhibiting or reducing fungi such as Candida, the effect of inhibiting or reducing pathogenic bacteria such as periodontal disease bacteria, the effect of inhibiting or reducing viruses such as herpesviruses, the effect of reducing or removing tongue coating composed of bacterial, fungal, and cellular secretions, food residues, and so on, the effect of suppressing or eliminating had breath, the effect of promoting salivary secretion and wetting the mouth, an anti-stress effect by giving a pleasant taste or an appropriate stimulation, the effect of giving a refreshing feeling by reducing a sticky feeling or the like, and an anti-inflammatory effect to ameliorate swelling or pain, and preferably means producing two or more of these effects.

As used herein, the term “the effect of promoting secretion of salivary adiponectin” means the effect of increasing the amount of adiponectin in saliva. The degree of the increase should not be restricted. Adiponectin is a protein secreted from fat cells. A reduction in the amount of secreted adiponectin can be a cause of insulin resistance, insulin responsiveness reduction, or metabolic syndrome development, and can cause hyperinsulinemia (an increase in the amount of secreted insulin in blood). A correlation has been found between the content of adiponectin and insulin in saliva and the content of them in blood. Therefore, the promotion of secretion of salivary adiponectin can prevent metabolic syndrome. It is also expected that the promotion of secretion of salivary adiponectin can improve the health of the oral environment.

### Advantageous Effects of the Invention

According to the present invention, the three specific ingredients (namely, cinnamon, low molecular proanthocyanidin, and menthol) are contained at the same time, which makes it possible to provide an oral composition and a confection that exhibit activity equivalent to or higher than that of a decanoic acid-containing composition.

### BRIEF DESCRIPTION OF THE DRAWINGS

FIG. 1 is a graph showing the results of improvement of the tongue symptom score when 2-fold dilutions of composition A and composition B are each administered in the evaluation f the effect of improving oral hygiene using an oral candidiasis mouse model (Example 1);

FIG. 2 is a graph showing the results of improvement of the tongue symptom score when 2-fold and 4-fold dilutions of composition B are each administered in the same evaluation (Example 1);

FIG. 3 is a graph showing changes in the amount of tongue coating of 13 volunteers before and after the intake of composition B in a volunteer test (Example 3);

FIG. 4 is a graph showing changes in the number of living Candida (C. albicans) cells in five Candida-positive volunteers before and after the intake of the composition in the same test (Example 3); and

FIG. 5 is a reaction strength image showing the amount of secreted salivary adiponectin m volunteers who took the composition of the present invention.

### DETAILED DESCRIPTION OF PREFERRED EMBODIMENTS

The oral composition of the present invention includes at least three ingredients including cinnamon, low molecular proanthocyanidin, and menthol thereinafter, also referred to as the specific ingredients in the present invention) as active ingredients. In addition to the specific ingredients, if necessary, the oral composition of the present invention may further contain at least one ingredient selected from a medium chain fatty acid, a medium chain fatty acid ester, and citral, and/or a thickener.

The cinnamon used in the present invention is a spice that contains cinnamaldehyde, an aromatic aldehyde having a phenylpropanoid skeleton, as an odorant. The cinnamon may be in the form of a cinnamon powder, a cinnamon oil, a cinnamon powder, or a cinnamon extract. Alternatively, the cinnamon may be cinnamaldehyde chemically synthesized or purified from to natural product or may be a cinnamaldehyde-containing composition (such as a cinnamaldehyde-containing flavoring agent or a cinnamaldehyde-containing oil).

The low molecular proanthocyanidin used in the present invention may be any material including, as main components, low molecular monomer, dimer, and trimer of proanthocyanidin. The low molecular proanthocyanidin is preferably Oligonol (registered trademark) commercially available from AMINO UP CHEMICAL Co., Ltd, (Sapporo, Hokkaido, Japan), which is a product of converting high molecular proanthocyanidin, a type of polyphenol contained in lychee fruit, to low molecular proanthocyanidin. Other examples of the low molecular proanthocyanidin include Pycnogenol (registered trademark, Horphag Research Ltd.), Flavangenol (registered trademark, TOYO SHINYAKU Co., Ltd.), etc., which may also be used.

The menthol used in the present invention is a colorless, volatile crystal with a peppermint scent. Menthol is not only widely used in oral compositions, confections, mouth refrigerants etc., but also in drugs. There are some isomers and derivatives of menthol. Natural products also contain such isomers and derivatives. Besides L-menthol used in the examples of the present invention, such isomers and derivatives chemically synthesized or extracted from natural products (e.g., menthol-containing flavoring agents) may also be used.

If necessary, a middle chain fatty acid may be used in the present invention. The middle chain fatty acid may be a linear or branched saturated fatty acid of 5 to 12 (preferably 8 to 12) carbon atoms, preferably a linear saturated fatty acid with the same number of carbon atoms. Examples of the middle chain fatty acid include pentanoic acid (also called valeric acid of 5 carbon atoms), hexanoic acid (caproic acid of 6 carbon atoms), heptanoic acid (enanthic acid of 7 carbon atoms), octanoic acid (caprylic acid of 8 carbon atoms), nonanoic acid (pelargonic acid of 9 carbon atoms), decanoic acid (capric acid of 10 carbon atoms), dodecanoic acid (lauric acid of 11 carbon atoms), and tetradecanoic acid (myristic acid of 12 carbon atoms).

If necessary, a middle chain fatty acid ester may be used in the present invention. The middle chain, fatty acid ester may be an ester of the middle chain fatty acid and a lower alcohol of 1 to 4 (preferably 1 to 2) carbon atoms. For example, when the middle chain fatty acid is decanoic acid, the middle chain fatty acid ester may be methyl decanoate, ethyl decanoate, propyl decanoate, or butyl decanoate. When the middle chain fatty acid is octanoic acid, the middle chain fatty acid ester may be methyl octanoate, ethyl octanoate, propyl octanoate, or butyl octanoate.

One of these middle chain fatty acids or middle chain fatty acid esters may be used alone, or two or more of these middle chain fatty acids and/or the middle chain fatty acid esters may be used in combination as needed. An essential oil (such as a decanoic acid-containing flavoring agent) purified from a natural product or a chemically synthesized product may also be used as the middle chain fatty acid and/or the middle chain fatty acid ester.

If necessary, citral may be used in the present invention. Citral is a monoterpene aldehyde, has a strong lemon scent, and is an essential oil component in herbs such as lemongrass, citrus fruits, Japanese pepper (Zanthoxylum), ginger, etc. A natural plant-derived essential oil (such as a citral-containing flavoring agent) may also be used as the citral, or chemically synthesized citral or citral purified from a natural product may also be used.

If necessary, a thickener may be used in the present invention. The thickener may be of any non-assimilable type, such as methyl cellulose propylene glycol, arabinoxylan, carrageenan, pectin, or xanthan gum. The addition of a non-assimilable thickener can extend the time for pathogenic organisms such as Candida to react with active ingredients other than the thickener, that the growth of pathogenic organisms can be more effectively inhibited.

The oral composition of the present invention includes the three specific ingredients: cinnamon, low molecular proanthocyanidin, and menthol, each in an amount necessary to improve oral hygiene.

As used herein, the term “an amount necessary to improve oral hygiene” means that when such an amount is used in the oral candidiasis mouse model shown in Example 1 below, it is observed that the tongue symptom score (0 to 4) is reduced to 3 or less (preferably 2.5 or less) or tends to be lower than that in the control (administration of water); when such an amount is used in the in vitro Candida mycelial growth inhibition test shown in Example 2 below, the ability to inhibit 50% or more of mycelial growth is observed; or when such an amount is used in the volunteer test shown in Example 3 below, at least one improvement effect selected from a reduction in the amount of tongue coating, a reduction in the number of living Candida cells, and a reduction in sticky feeling in the mouth is observed.

The oral candidiasis mouse model is preferably used because objective quantification can be performed in it.

The oral composition of the present invention may contain cinnamon, low molecular proanthocyanidin and menthol in such amounts that when the composition is administered to the mouth, the concentrations of cinnamon, low molecular proanthoeyanidin, and menthol can reach 70 μg/mL or more, 125 μg/mL or more, and 50 μg/mL of more, respectively, at a local site where the effect is to be expected (e.g., a site on the oral mucosa, such as a lingual or sublingual site).

More specifically, the oral composition of the present invention may contain 0.28 mg or more, preferably 0.56 mg or more, more preferably 1.12 mg or more, even more preferably 2.24 mg or more, further more preferably 5.6 mg or more, still more preferably 11.2 mg or more of cinnamon per one dosage (hereinafter referred to as unit dosage).

The oral composition of the present invention may contain, for example, 0.018 mg or more, preferably 0.036 mg or more, more preferably 0.072 mg or more, even more preferably 0.14 mg or more, further more preferably 0.36 mg or more, still more preferably 0.72 mg or more of cinnamaldehyde per unit dosage.

The oral composition of the present invention may contain, for example, 0.5 mg or more, preferably 1.0 mg or more, more preferably 2.0 mg or more, even more preferably 4.0 mg or more, further more preferably 10.0 mg or more, still more preferably 20.0 mg or more of low molecular proanthocyanidin per unit dosage.

The oral composition of the present invention may contain, for example, 0.2 mg or more, preferably 0.4 mg or more, more preferably 0.8 mg or more, even more preferably 1.6 mg or more, further more preferably 4.0 mg or more, still more preferably 8.0 mg or more of menthol (e.g., L-menthol) per unit dosage.

The oral composition of the present invention may contain, for example, 0.008 mg or more, preferably 0.016 mg or more, more preferably 0.032 mg or more, even more preferably 0.064 mg or more, further more preferably 0.16 mg or more, still more preferably 0.32 mg or more of a middle chain fatty acid (e.g., decanoic acid) or a middle chain fatty acid ester per unit dosage.

The oral composition of the present invention may contain, for example, 0.14 mg or more, preferably 0.28 mg or more, more preferably 0.56 mg or more, even more preferably 1.12 mg or more, further more preferably 2.8 mg or more, still more preferably 5.6 mg or more of citral per unit dosage.

The oral composition of the present invention may contain, for example, 0.09 mg or more, preferably 0.18 mg or more, more preferably 0.36 mg or more, even more preferably 0.72 mg or more, further more preferably 1.8 mg or more, still more preferably 3.6 mg or more of a thickener (e.g., methyl cellulose) per unit dosage.

The upper limit of the content of each of these ingredients may be appropriately determined taking into account, for example, an adverse effect such as interference of taste or excessive stimulation, the content of other coexisting ingredients, and cost effectiveness. For example, the total weight of these active ingredients may make up 99% or less (in one mode), 90% or less (in another mode), 80% or less (in a further mode), or 50% or less (in a still further mode) of the weight of the oral composition of the present invention.

The oral composition of the present invention may be in any dosage form as long as it can be effective for oral hygiene when held for a certain period of time in the mouth regardless of whether it is swallowed or spit out after the holding in the mouth. Examples of the dosage form include common drug dosage forms (such as a gargle, a liquid formulation, an ointment, and a cleaning agent) and other forms such as confections, cosmetics, and oral hygiene products. The cosmetics and oral hygiene products include, for example, mouthwashes, dentifrices, gargles, gels, and sprays. In addition to the active ingredients, each of these dosage forms may contain a known additive, used in each dosage form, such as a known carrier, diluent, or auxiliary material. The oral composition of the present invention is preferably used in the form of a confection. In particular, the oral composition of the present invention is preferably formed into a candy piece, a chewing gum, a troche, a gummy candy piece, or a tablet so that it can be held and dissolved in the mouth.

(Confection Mode)

The oral composition of the present invention may be produced as a confection. In this case, the oral composition of the present invention is preferably formed into a candy piece, a chewing gum, a troche, a gummy candy piece, or a tablet so that it can be held and dissolved in the mouth. The confection that can be held and dissolved in the mouth may also be a confectionery product, a soft candy piece, a tablet-shaped candy piece, a gummy candy piece, a jelly or gel-like semi-solid agent, or a viscous liquid preparation. In a preferred mode, the oral composition of the present invention is a candy-like preparation that contains a thickener as an ingredient and has a water content of 5% by weight or less so that it can remain or stay particularly for a long time in the mouth. In another preferred mode, the oral composition of the present invention is a gel preparation that can be spread, applied or attached into the mouth using a spray device or an applicator. Besides the above confections, a beverage, a yogurt, a dressing, or a supplement may also be an example of the form.

For the purpose of improving oral hygiene, non-sugar orally-dissolving preparations based on a sugar alcohol such as maltitol or palatinose and being free of natural sugars are preferable to sugar-based candy products because such preparations are less likely to cause dental caries. The taste of the composition is not restricted, and a mint candy taste or any of various other tastes such as lemon and milk tastes may be imparted to the composition. A suitable taste may be selected depending on the purpose. The content of each active ingredient may also be freely adjusted to such a level that consumers can take the desired amount of each active ingredient without any trouble.

### EXAMPLES 1 TO 3

Hereinafter, the present invention will be more specifically descried with reference to Examples 1 to 3, which, however, are not intended to limit the scope of the present invention.

### EXAMPLE 1

### Evaluation of the Oral Hygiene-Improving Effect Using Oral Candidiasis Mouse Model

Two viscous liquid compositions (candy) were each prepared as an oral composition according to the present invention by adding the amount of each additive shown in Table 1 and a sweetener and an emulsifier(each 0.1% based on the weight of the composition) to reduced maltose syrup (67.2% based on the weight of the composition) and reduced sugar syrup (31.3% based on the weight of the composition). The content of each additive shown in Table 1 is in units of additive weight per 1 mL of the composition (mg/mL).

The additives shown in Table 1 were used which include cinnamon powder (S&B Foods Inc.), Oligonol (AMINO UP CHEMICAL Co., Ltd), L-menthol (Osaka-Koryo Corporation), decanoic acid-containing flavoring, agent (V. MANE FILS JAPAN, LTD.), citral-containing flavoring agent (V. MANE FILS JAPAN, LTD.), and methyl cellulose (Shin-Etsu Chemical (o., Ltd.). The cinnamon powder used at this time had a cinnamaldehyde content of 6.4%.

TABLE 1

| | Composition A | Composition B |
|---|---|---|
| Cinnamon powder | 2.8 mg/ml | 2.8 mg/ml |
| Oligonol | 5.0 mg/ml | 5.0 mg/ml |
| L-menthol | 2.0 mg/ml | 2.0 mg/ml |
| Decanoic acid | — | 0.08 mg/ml |
| Citral | — | 1.4 mg/ml |
| Methyl cellulose | 0.9 mg/ml | 0.9 mg/ml |

ICR mice (female, 6 weeks old) were subcutaneously injected with 100 mg/kg of prednisolone for immunosuppression one day before inoculated with Candida cells (Candida albicans strain TIMM1768 stored in Teikyo University Institute of Medical Mycology). From the day, the mice were allowed to freely take tap water containing 4.125 mg/mL of chlortetracycline hydrochloride. On the day of the inoculation, the mice were sedated by previous intramuscular administration of 14.4 mg/kg of chlorpromazine hydrochloride. A cotton swab was dipped in a Candida cell suspension with an adjusted cell concentration of 2×10⁸ cells/mL and then rubbed against the oral cavity of the mice to be inoculated. The composition to be evaluated was diluted 2- or 4-fold with sterilized water. Three hours, 24 hours, and 27 hours after the inoculation with Candida cells. 50 μL of the dilution was administered dropwise to the dorsum of the tongue of the mice using a gastric tube for mice. Sterilized water was administered as a control. Two days after the inoculation, the mice were euthanized, and the tongue symptom score (0 to 4) was evaluated according to the criteria shown in Table 2.

TABLE 2

| Score | Symptom |
|---|---|
| 0 | Normal (no fur is observed on the tongue surface.) |
| 1 | Fur is observed on a 19% or less area of the tongue surface. |
| 2 | Fur is observed on a 20% to 89% area of the tongue surface. |
| 3 | Fur is observed on a 90% or more area of the tongue surface. |
| 4 | Thick fur is observed on a 90% or more area of the tongue surface. |

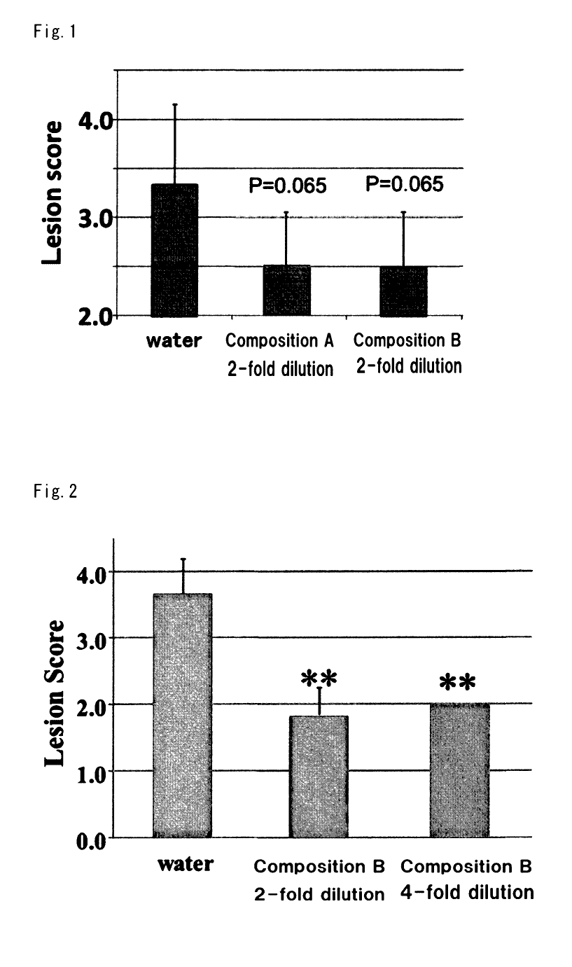

FIG. 1 shows the results of the administration of a 2-fold dilution of each of compositions A and B. FIG. 2 shows the results of the administration of a 2-fold dilution of composition B and a 4-fold dilution of composition B.

FIG. 1 shows that as compared with the case where only water was administered, the tongue symptom tended to be ameliorated in the case where a 2-fold dilution of composition A or B according to the present invention was administered, although no significant difference was observed. In particular, composition A neither contains decanoic acid, which has been considered to be a promising candidate for anti-Candida drugs, nor citral, which is known to show a synergistic effect when used in combination with decanoic acid. Nevertheless, composition A surprisingly showed the same level of ameliorating effect as composition B containing these materials.

The 2-fold dilution of composition A has a cinnamon concentration of 1.4 mg/ml, and an Oligonol concentration of 2.5 mg/mL (each ½ of the content shown in Table 1). The conventionally known effective concentrations of these materials administered alone were 19.5 mg/mL (Taguchi Y, et al. Med Mycol J. 2011:52;145-152) and 20 mg/mL (Hayama K. et al. Jpn J Med Mycol. 2010;51:137-142), respectively. It was therefore found that a sufficient ameliorating effect can be obtained using a much smaller amount of these materials. In particular, Oligonol has been considered to be difficult to use in an oral composition because it has strong astringency and can significantly deteriorate taste when its concentration is as high as 20 mg/mL. Therefore, the fact that the ameliorating effect can be obtained with a smaller amount is very important for practical use.

On the other hand, FIG. 2 shows that as compared with the case where only water was administered, the score value was reduced to about 2 and a significant tongue symptom ameliorating effect was observed in the case where a 2- or 4-fold dilution of composition B according to the present invention was administered.

These results suggest that the composition of the present invention tends to inhibit Candida cells on the tongue from forming fur and to ameliorate the symptoms and can improve the oral environment or maintain the oral hygiene.

### EXAMPLE 2

### Evaluation of Anti-Candida Activity by In Vitro Candida mycelial Growth Inhibition Test

The Candida mycelial growth inhibition activity of composition A prepared in Example 1 was evaluated by crystal staining method. In this method, Candida cells (Candida albicans strain TIMM1768 stored in Teikyo University Institute of Medical Mycology) growing in a mycelial form are stained with a crystal violet solution, and the amount of the staining is used as an index of the degree of Candida mycelial growth based on the fact that the amount of the staining correlates with the number of grown colonies or the amount of intake of [H3]-glucose into cells. Living Candida cells prepared on a fetal calf serum (FCS)-containing RPMI1640 medium were dispensed at a concentration of 5×10² cells/well into a 96-well flat bottom plate. Composition A was diluted to each concentration. Each dilution of composition A was added to the plate and subjected to culturing under the conditions of 37° C. and 5% CO₂ for about 20 hours. Subsequently, after the Candida mycelia on the well bottom were stained with a crystal violet solution, the crystal violet was extracted with an isopropyl alcohol solution from the stained Candida cells. The amount of the dye was spectroscopically measured (620 nm) to determine the inhibitory amount (relative value).

As a result of the test, compound A was found to have the ability to inhibit 50% of mycelial growth even when diluted about 40 times. Table 3 shows the concentration of each active ingredient in the 40-fold dilution of composition A.

This result suggests that the composition can inhibit the mycelial growth of Candida, which is a cause of tongue coating that can deteriorates oral hygiene, so that the adhesion of Candida cells to the oral mucosa can be prevented, which can be effective in maintaining oral hygiene. The same test was performed using composition B prepared in Example 1. As a result, composition B was found to have the ability to inhibit 50% of mycelial growth even when diluted about 1,000 times.

TABLE 3

| | Composition A |
|---|---|
| Cinnamon powder | 70 μg/ml |
| Oligonol | 125 μg/ml |
| L-menthol | 60 μg/ml |
| Methyl cellulose | 22.5 μg/ml |

### EXAMPLE 3

### Evaluation of the Oral Hygiene-Improving Effect Using Volunteer Test

A viscous liquid composition was prepared as an oral composition according to the present invention by adding the amounts of Oligonol and methyl cellulose for composition B shown in Table 1 and a sweetener and an emulsifier (each 0.1% based on the weight of the composition) to reduced maltose syrup (67.2% based on the weight of the composition) and reduced alga syrup (31.3% based on the weight of the composition). The viscous liquid composition was concentrated by boiling until its water content reached 0.5%. The amounts of cinnamon powder, L-menthol, decanoic acid, and citral for composition B shown in Table 1 were further added to the concentrate. The mixture was kneaded and then formed into 4 g pieces of candy.

Volunteers ate the prepared candy pieces (composition B) for 2 days. The amount of tongue coating, the number of living Candida (C. albicans) cells, and the effect of improving oral hygiene (questionnaire) were evaluated before and after the intake. Although containing low molecular proanthocyanidin with strong astringency, the candy did not have any particular unpleasant astringency. Immediately after getting up in the morning of the first day of the test, the volunteers rubbed their tongue three times with a commercial tongue brush and then put the brush into a tube containing a saline solution. The sample was transferred in cold storage. After the sample was centrifuged, the precipitate was collected and weighed. The measured weight was used as the amount of tongue coating. The precipitate was also thoroughly dispersed and re-suspended in an RPMI1640 medium. The suspension was applied to a Candida selective medium and cultured at 37° C. for 3 days before the number of living Candida cells was counted. The volunteers ate about 4 g (one piece of the candy) of composition B after each meal, three times a day for two days. Table 4 shows the content of each active ingredient in one piece of the candy. In the morning of day 3, the tongue coating, was collected and transferred in the same manner, and the amount of tongue coating, and the number of living Candida cells were measured. By filling out a questionnaire, the volunteers also rated, on a scale of 1 to 5, any changes in oral physiological conditions before and after the intake of the composition.

TABLE 4

| | Composition B |
|---|---|
| Cinnamon powder | 11.2 mg |
| Oligonol | 20.0 mg |
| L-menthol | 8.0 mg |
| Decanoic acid | 0.32 mg |
| Citral | 5.6 mg |
| Methyl cellulose | 3.6 mg |

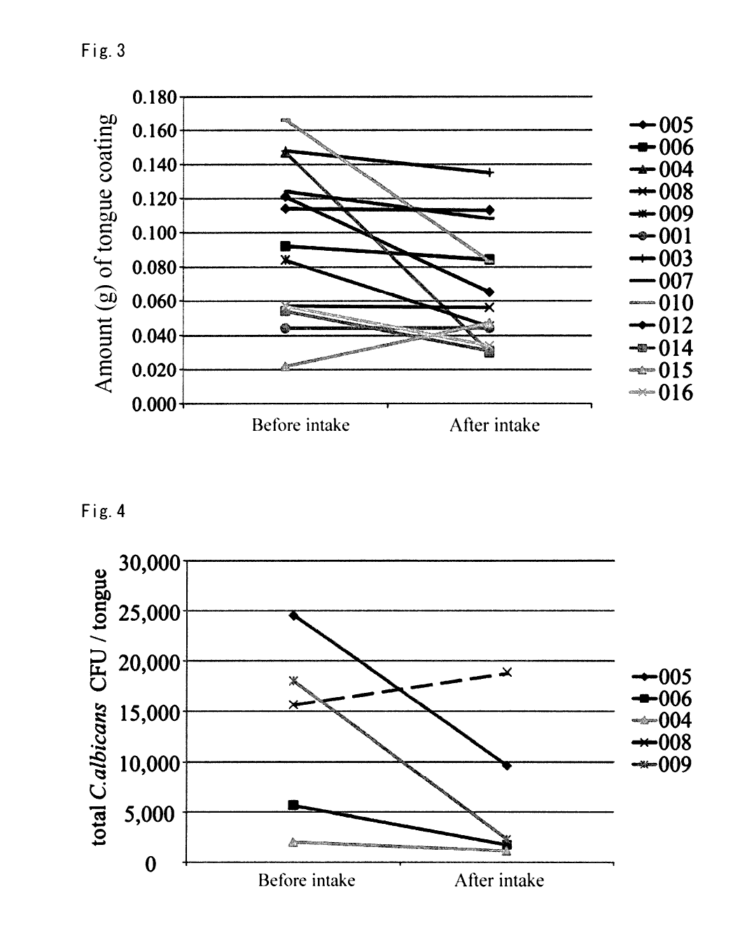

FIG. 3 shows changes in the amount of tongue coating of 13 volunteers before and after the intake of composition B. The results of the test show that the amount of tongue coating, which includes separated epithelial cells, bacteria, fungi, food residues, etc., decreases after the intake of the composition.

FIG. 4 shows changes in the number of living Candida (C. albicans) cells in five Candida-positive volunteers, from whom colonies were detected with the Candida selective medium. FIG. 4 shows that in four of the five volunteers, the number of Candida (C. albicans) cells in the tongue coating decreases after the intake of the composition. The one volunteer, who did not show this decrease, showed a decrease in the number of cells of a Candida species (Candida sp.) other than C. albicans (before the intake: 2,970 CFU/tongue, after the intake: 1,452 CFU/tongue).

Table 5 shows changes in the oral physiological conditions of the 13 volunteers before and after the intake of the composition. The results show that the effect of reducing sticky feeling in the mouth is high for 8 volunteers at least 50 years old.

It has been found from the results that the composition of the present invention is effective as an oral composition for reducing the amount of tongue coating, the number of living Candida cells, and sticky feeling in the mouth, and can be used in humans.

TABLE 5

| | | | | | Odd | | | | |
|---|---|---|---|---|---|---|---|---|---|
| | | Bad | Reduction of sticky | Dry | feeling on | Ease of | Improved | Itchy feeling | Bowel |
| | | breath | feeling in mouth | mouth | tongue | eating | gustation | in throat | movement |
| Male 9 | 50.0 | 2.9 | 3.6 ± 1.01 | 2.9 | 3.2 | 2.9 | 2.9 | 2.8 | 2.9 |
| Female 4 | 47.5 | 3.3 | 3.5 ± 0.58 | 3.0 | 2.3 | 2.8 | 3.0 | 3.3 | 3.3 |

| | | | | | | | | |
|---|---|---|---|---|---|---|---|---|
| Ave. | 3.1 | 3.5 ± 0.88 | 2.9 | 2.7 | 2.8 | 2.9 | 3.0 | 3.1 |
| Age 50 or older | 3.1 | 3.9 ± 0.64 | 3.0 | 3.0 | 2.9 | 3.0 | 2.9 | 3.0 |
| “8 volunteers: | | | | | | | | |
| Male 6, Female 2” | | | | | | | | |

5 absolutely yes
4 yes
3 may be yes, may be no
2 no
1 absolutely no

(Measurement of Salivary Adiponectin)

Volunteers who ate the candy of Example 3 were subjected to the measurement of salivary adiponectin. The measurement method was as follows. After the measurement sample was denatured with ethanol, the band image of the saliva sample was obtained by Western blotting method under the conditions of ethanol denaturation.

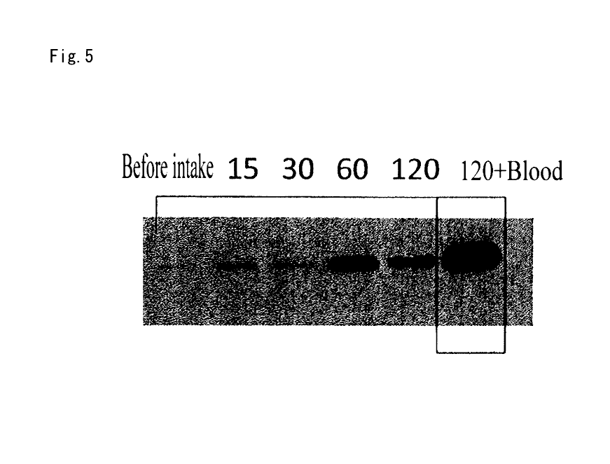

Specifically, saliva samples were collected from two volunteers before they ate the candy of Example 3 (doughnut shape 20 mm in large diameter, 7 mm in hole diameter) and 15 minutes, 30 minutes, 60 minutes, and 120 minutes after they ate the candy. When saliva samples were collected, saliva was directly collected into sampling tubes through a straw. Each collected sample was centrifuged and then cryopreserved. Subsequently, 2-mercaptoethanol was added to the thawed sample, which was then denatured by heating at 95° C. for 10 minutes. The sample was then separated by SDS-PAGE electrophoresis with polyacrylamide (3 to 8%). The bands were transferred to a PVDE membrane and then allowed to react with a monoclonal antibody to detect an image indicating reaction strength (FIG. 5).

(Amount of Secreted Adiponectin in Each Sample)

FIG. 5 shows an example of the band image of the saliva sample obtained by Western blotting method under denaturation conditions. The denaturation conditions were addition of 2-mercaptoethanol and heating at 95° C. for 10 minutes. The anti-adiponectin antibody used was a mouse IgG1 antibody MAB3604 from Millipore. The antibody reaction with adiponectin in the saliva sample gradually appeared 15 minutes after the intake of the candy of Example 3 and became significant 60 minutes or more after the intake. This suggests that the intake of the candy of Example 3 is effective in promoting the secretion of salivary adiponectin in the mouth. This also suggests that the amount of secreted salivary adiponectin increases with the time during which the candy of Example 3 is absorbed into the body.

[Periodontal Disease Prevention]

As oral health deteriorates, the risk of periodontal disease increases. As the life expectancy increases, periodontal disease becomes a cause of losing teeth, American Dental Association reports that in the U.S. 80% of adults 65 years and older have periodontal disease. A further problem is that periodontal disease has an important effect on diabetes, respiratory diseases, pregnancy troubles, and heart diseases.

Periodontal disease prevention is a measure against metabolic syndrome. Periodontal disease is a factor having an adverse effect on diabetes, a cause of metabolic syndrome. Since periodontal disease has few subjective symptoms, bacteria are insidiously transferred from the teeth to the rest of the body. Nowadays, it is pointed out that there is a relationship between metabolic syndrome and periodontal disease. Substances called cytokines play an important role in that mechanism. Cytokines, which are proteins produced by lymphocytes and so on, act to maintain homeostasis. However, when excessively produced due to bacterial infection or the like, cytokines can negatively act on the living body. Antibacterial proteins such as lysozyme, histatin, and cystatin in saliva help to prevent infection by suppressing the proliferation and toxicity of bacteria and modulating the immune system.

Also in view of the prevention of periodontal disease, salivary adiponectin, which is directly secreted from salivary glands, would be useful as an oral health marker.

Each of the above examples shows, as examples of dosage form, common drug dosage forms (such as a gargle, a liquid formulation, an ointment, and a cleaning agent) and other forms such as confections, cosmetics, and oral hygiene products. Among them, non-pharmaceutical, oral compositions (such as confections, cosmetics, and oral hygiene products) are not used directly in medical practices or regenerative medical practices using biological materials.

In each of the above examples, adiponectin in human saliva is identified or evaluated. However, adiponectin in human saliva, is a non-limiting example. Saliva samples may also be collected from various other organisms with salivary glands (such as pets or domestic animals), and adiponectin in the saliva of such various organisms may also be identified, quantified, or evaluated.

(Method for Producing Oral Composition)

An example of the method of the present invention for producing an oral composition will be described below. This production method may be directly used as a method for producing a tablet-type confection that can be held and dissolved in the mouth.

The method of the present invention for producing an oral composition includes the following steps of

(1) dissolving at least 0.2 to 1.0% by weight of low molecular proanthocyanidin in 0.2 to 2.0% by weight of at least one water-soluble solvent selected from glycerin or propylene glycol to form a solution (polyphenol solution), wherein the low molecular proanthocyanidin is a polyphenol soluble in the water-soluble solvent (dissolving step);

(2) kneading at least 0.1 to 0.5% by weight of cinnamon and 0.1 to 0.4% by weight of menthol into sugar-free, candy- or gel-like, base dough to prepare candy- or gel-like kneaded dough (preparing step); and

(3) mixing the solution with the kneaded dough (mixing step).

(Dissolving Step)

First, at least 0.2 to 1.0% by weight of low molecular proanthocyanidin as a polyphenol soluble in a water-soluble solvent is dissolved in 0.2 to 2.0% by weight of at least one water-soluble solvent selected from glycerin or propylene glycol to form a solution (the dissolving step).

(Water-Soluble Solvent)

Examples of the water-soluble solvent used in the present invention include glycerin, propylene glycol, and a mixture thereof, which are food additives. Glycerin and propylene glycol are water-soluble solvents having similar physiochemical properties. The water-soluble solvent is used to dissolve the polyphenol, which is soluble in the water-soluble solvent, at a stage where non-sugar candy is produced. In the present invention, the water-soluble solvent may contain up to 10% (v/v) of water. In general, glycerin, propylene glycol, or a mixture thereof may be used as it is. Alternatively, up to 10% (v/v) of water may be added to any of these solvents in order to increase the solubility of the polyphenol in the water-soluble solvent. It should be noted that if more than 10% (v/v) of water is added, the final kneaded dough can have an increased water content, which can have an adverse effect on the quality of the candy, such as long-term stability, and thus is not preferred. For a taste balance and handleability, a certain concentration of propylene glycol or propylene glycol alone may be used. In particular, propylene glycol has viscosity lower than that of glycerin and thus is effective in increasing handleability and the rate of dissolution. However, glycerin is a preferred water-soluble solvent because it is available at relatively low cost and has a favorable taste.

As used herein, the term “polyphenol soluble in a water-soluble solvent” refers to a polyphenol soluble in any water-soluble solvent from glycerin, propylene glycol, or a mixture thereof.

For example, some polyphenols soluble in the water-soluble solvent are completely dissolved or form scattered or suspended fine solid particles when a solid of each of these polyphenols is mixed into the water-soluble solvent in an amount 20 times the weight of the polyphenol and the mixture is visually observed at 37° C. after 48 hours. The term “polyphenol soluble in the water-soluble solvent” is intended to include all of such polyphenols.

On the other hand, when a solid of a polyphenol hardly-soluble in the water-soluble solvent is mixed into the water-soluble solvent in an amount 20 times the weight of the polyphenol and the mixture is visually observed at 37° C. after 48 hours, the solid undergoes almost no change and remains as before the mixing.

Examples of a polyphenol soluble in all of glycerin, propylene glycol, and a mixture thereof include a lychee-derived polyphenol, an evening primrose-derived polyphenol, a oolong tea-derived polyphenol, a green tea-derived polyphenol, a blackberry leaf tea-derived polyphenol, and tannic acid extracted and purified from tea leaves, persimmons, lotus roots, coffee beans, perillas, gallnuts, gall nuts, Geranium herb, saxifrage, Japanese quinces, camellias, rosemary, etc.

The lychee-derived polyphenol may be, for example, Oligonol (registered trademark), Oligonol is a lychee-derived polyphenol preparation developed, produced, and sold by AMINO UP CHEMICAL Co., Ltd. Oligonol includes a low molecular polyphenol produced using lychee and catechin as raw materials.

The evening primrose-derived polyphenol may be, for example, a product named “Oseran” (registered trademark) sold by Nagaoka Perfumery Co., Ltd.

The oolong tea-derived polyphenol may be, for example, a product named “Oolong Tea Extract” manufactured by MARUZEN PHARMACEUTICALS CO., LTD.

The green tea-derived polyphenol may be, for example, any of a series of products named “Sunpherion” (registered trademark) manufactured by Taiyo Kagaku Co., Ltd.

The blackberry leaf tea-derived polyphenol may be, for example, a product named “Blackberry Leaf Tea Extract” manufactured by MARUZEN PHARMACEUTICALS CO. LTD.

Tannic acid may be, for example, a product named “Tannic Acid AL” manufactured by Fuji Chemical Industry Co., Ltd.

All of these polyphenols soluble in the water-soluble solvent are allowed to be used in food products and available as food raw materials or food additives.

Particularly in view of easy solubility in the water-soluble solvent, the lychee-derived polyphenol, the evening primrose-derived polyphenol, the green tea-derived polyphenol, the blackberry leaf tea-derived polyphenol, and tannic acid are preferred, and Oligonol (registered trademark) is more preferred.

Polyphenols, which are soluble in the water-soluble solvent and may be used in the present invention, include a certain polyphenol such as resveratrol which is hardly soluble in glycerin but soluble in propylene glycol. Specifically, low molecular proanthocyanidin is used in the present invention. When resveratrol is used as the polyphenol, propylene glycol needs to be used as the water-soluble solvent.

In the present invention, low molecular proanthocyanidin, which is the polyphenol soluble in the water-soluble solvent, is previously dissolved in the water-soluble solvent, so that the polyphenol can be mixed without being in the form of a powder. Therefore, the polyphenol is prevented from forming a lump in the kneaded dough, and the resulting candy can have reduced graininess in the mouth. When glycerin is used as the solvent, a higher content of the polyphenol can be achieved, and the bitterness and astringency of the polyphenol can also be suppressed.

The low molecular proanthocyanidin, as the polyphenol soluble in the water-soluble solvent, may be dissolved in the water-soluble solvent in an amount from once to twice the weight of the low molecular proanthocyanidin. Preferably, the low molecular proanthocyanidin is dissolved in the water-soluble solvent in an amount from once to 1.5 times the weight so that the influence of the typical taste of the water-soluble solvent can be reduced. The solubility of the low molecular proanthocyanidin in the water-soluble solvent may be such that it can be almost dissolved in the end, specifically, such that at least 70% by weight of the polyphenol solid can be dissolved. If the solubility is less than 70% by weight, the low molecular proanthocyanidin can cause the final non-sugar candy to maintain strong bitterness or astringency and to have significant graininess, which is not preferred.

The low molecular proanthocyanidin is mixed with the water-soluble solvent and dissolved by stirring or the like until the above solubility is reached, so that a solution (polyphenol solution) is obtained. In this dissolving process, heating at 60° C. or less may be performed so that the thermal denaturation of the low molecular proanthocyanidin can be avoided as much as possible, or several tens of hours may be spent for the dissolution. The low proanthocyanidin may also be allowed to stand without stirring, as long as it can be dissolved in the water-soluble solvent. An acid or a base may also be added and mixed to adjust the pH and promote the dissolution. In this case, however, it should be checked with an analyzer such as a high-performance liquid chromatograph that the low molecular proanthocyanidin is not denatured or decomposed.

(Preparing Step)

Next, at least 0.1 to 0.5% by weight of cinnamon and 0.1 to 0.4% by weight of menthol are kneaded into sugar-free, candy- or gel-like, base dough to prepare candy- or gel-like kneaded dough, is concentrated by boiling (the preparing step). In the preparing step, the boiling method may include boiling a carbohydrate solution containing one or more carbohydrates until its water content reaches 5% by weight or less according to a conventional method. When the carbohydrate solution is prepared, if necessary, water may also be added. The boiling temperature may be, for example, 120 to 160° C. although it may be set according to a conventional method.

(Mixing Step)

Subsequently, the solution (polyphenol solution) is mixed with the kneaded dough concentrated by boiling (the mixing step). The method of mixing the solution (polyphenol solution) with the kneaded dough may be, but not limited to, a method that includes adding the solution (polyphenol solution) to the kneaded dough and kneading them, which may be specifically either a batch process or a continuous process. For example, the batch process may include adding a certain amount of the solution (polyphenol solution) to the kneaded dough and sufficiently mixing them. For example, the continuous process may include mixing the kneaded dough and the solution (polyphenol solution) in a constant ratio while feeding them using constant rate pumps or the like. In all cases, the solution (polyphenol solution) is added in such an amount that the non-sugar candy can finally have a polyphenol content of 0.2 to 1.0% by weight.

The mixing temperature may be, for example, 120 to 140° C. so that high performance mixing can be achieved. The water-soluble solvent used herein is not volatile under such temperature conditions. Therefore, the final concentration of the water-soluble solvent in the non-sugar candy is from 0.2 to 2.0% by weight.

(Other Kneading Methods)

Alternatively, the method of preparing the kneaded dough may include mixing the solution (polyphenol solution) into a carbohydrate solution and concentrating the mixture by boiling. In this method, however, heat at 120° C. or more can be applied for a long time to boil and concentrate the carbohydrate solution until the desired water content is reached, and therefore, attention should be paid to the possibility that the polyphenol may be denatured in the resulting confection. In the present invention, however, the solution (polyphenol solution) is mixed with the kneaded dough concentrated by boiling as mentioned above. This mixing process can be performed during the process of cooling the kneaded dough, so that no or little denaturation of the polyphenol will occur, which raises no problem.

(Shaping Method)

The kneaded dough obtained in this way may be shaped by depositing or stamping after a certain period of cooling. These processes may be performed according to conventional candy making techniques.

(Method for Adding Additives)

If necessary, the confection may contain seasoning ingredients, colorants, flavoring agents, other functional ingredients such as dietary fibers, or other additives. These materials may be added in the preparation process, may be added simultaneously with the solution (polyphenol solution), or may be added at the final stage. The timing of the addition may be determined in a conventional way depending on the properties of the raw materials and the characteristics of the manufacturing line.

The confection obtained in this way contains the polyphenol in such an amount that the function of the polyphenol can be expected. In the confection, however, the bitterness and astringency of the polyphenol are reduced, and graininess or the like is not perceived. Therefore, the confection is easy to take, is suitable as a functional food product that can be taken continuously or conveniently, and can be stored for a long time. In addition, the polyphenol-containing confection obtained by the above method has long-lasting physical properties. In particular, the lychee-derived low molecular polyphenol provides a good polyphenol impression. According to the above method, the confection can be efficiently produced by as manufacturing process suitable for commercial production.

(Non-Sugar Candy Mode)

In an embodiment of the present invention, the confection is obtained as a non-sugar candy product that contains 0.2 to 2.0% by weight of at least one water-soluble solvent selected from glycerin or propylene glycol and 0.2 to 1.0% by weight of the polyphenol soluble in the water-soluble solvent.

The term “non-sugar candy” refers to candy in which the content of saccharides is 0.5% or less based on the total weight of one piece of the preparation, specifically, the total amount of not only sugar, but also monosaccharides and disaccharides, such as fructose, glucose, and maltose, is 0.5 g or less per 100 g of the candy, and the water content is adjusted to 5.0% or less. To form the non-sugar candy of the present invention, what is called a sugar alcohol, such as reduced maltose syrup (maltitol), reduced sugar syrup, reduced palatinose, xylitol, erythritol, lactitol, mannitol, or sorbitol is used as a carbohydrate material for the dough to be kneaded instead of monosaccharides and disaccharides. These carbohydrates may be used alone, or two or more sugar alcohols may be mixed in a desired ratio depending on, for example, the desired physical properties or the product concept.

In the present invention, the sugar alcohol for use as the dough to be kneaded is not intended to include the water-soluble solvent. In the present invention, however, the water-soluble solvent may also be used as an ingredient for the dough to be kneaded as long as its content falls within the above range.

### EXAMPLES 4 to 9

Hereinafter, the present invention will be more specifically described with reference to Examples 4 to 9, which however are not intended to limit the present invention. Hereinafter, “%” and “parts” means “% by weight” and “parts by weight,” respectively, unless otherwise stated.

<Solubility Test>

The different polyphenols shown in Table 6 were tested for solubility in glycerin and propylene glycol. Each powdered polyphenol was mixed with glycerin or propylene glycol in an amount 20 times the weight of the polyphenol. The mixture was heated at 37° C. for 48 hours with stirring at the appropriate times. As a result, the polyphenols were classified into those soluble in the water-soluble solvent and those hardly-soluble in the water-soluble solvent.

The criteria shown below were used to determine whether the polyphenol is “soluble” or “hardly soluble.”

Soluble: After 48 hours at 37° C., the polyphenol is dissolved to such an extent that the solid of the polyphenol is not visually observed or significantly reduced in the solution as compared with the state before the mixing. In particular, the case where the solid is not visually observed is expressed as “easily soluble.”

Hardly soluble: After 48 hours at 37° C., the solid of the polyphenol in the solution is visually observed and almost unchanged from before the mixing.

TABLE 6

| | | Solubility | |
|---|---|---|---|
| Sample name | | Glycerin | Propylene glycol |
| Lychee-derived low molecular | AMINO UP | Easily soluble | Easily soluble |
| polyphenol (“Oligonol”) | CHEMICAL Co., Ltd. | | |
| Tannic Acid AL | Fuji Chemical Industry | Easily soluble | Easily soluble |
| | Co., Lid. | | |
| Evening primrose polyphenol | Nagaoka Perfumery | Easily soluble | Easily soluble |
| (“Oseran”) | Co., Ltd. | | |
| Oolong Tea Extract | MARUZEN | Easily soluble | Easily soluble |
| | PHARMACEUTICALS | | |
| | CO., LTD. | | |
| Resveratrol | Techno Science Co., | Hardly soluble | Easily soluble |
| | Ltd. | | |
| Quercetin | Medience Corporation | Hardly soluble | Hardly soluble |
| “Sunphenon” 90S | Taiyo Kagaku Co., Ltd. | Easily soluble | Easily soluble |
| Blackberry Leaf Tea Extract | MARUZEN | Easily soluble | Easily soluble |
| | PHARMACEUTICALS | | |
| | CO., LTD. | | |

In the table, the names are all trade names, and each name in quotation marks is a registered trademark.

### EXAMPLE 4

In a vacuum kettle, reduced maltose syrup was concentrated by boiling until its water content reached 5% by weight or less, so that dough to be kneaded was obtained. Oligonol was dissolved in glycerin in an amount equal to the weight of Oligonol. The Oligonol solution was added in an amount of 1.5% by weight to the dough, and they were mixed. In this process, 0.1% by weight of a herb flavor was added. The resulting kneaded dough was formed into 4 g spherical pieces by stamping. In this way, herb-flavored, non-sugar candy pieces with a water content of 2% by weight were obtained. The resulting, polyphenol-containing, non-sugar candy had no graininess in the mouth and had a mild taste with the typical bitterness and astringency of the polyphenol kept low.

### EXAMPLE 5

In a vacuum kettle a carbohydrate solution containing dissolved reduced palatinose was concentrated by boiling until its water content reached 5% by weight or less, so that dough to be kneaded was obtained. Oligonol was dissolved in glycerin in an amount twice the weight of Oligonol. The Oligonol solution was added in an amount of 3% by weight to the dough, and they were mixed. In this process, 0.1% by weight of a menthol flavor and 0.1% by weight of vitamin C were added. The resulting kneaded dough was formed into 4 g fiat pieces by casting. In this way, herb-flavored, non-sugar candy pieces with a water content of 2% by weight were obtained. Although having the typical bitterness and astringency of the polyphenol, the resulting, polyphenol-containing, non-sugar candy had no graininess in the mouth and was comfortably edible. Although the candy began to become sticky, slightly fast, the storage stability of the candy was at an acceptable level for confectionery products.

### EXAMPLE 6

In a vacuum kettle, reduced maltose syrup was concentrated by boiling until its water content reached 5% by weight or less, so that dough to he kneaded was obtained. Oligonol was dissolved in glycerin in an amount equal to the weight of Oligonol. The Oligonol solution was added in an amount of 0.4% by weight to the dough, and they were mixed. In this process, 0.1% by weight of a lemon flavor was added. The resulting kneaded dough was formed into 4 g barrel-shaped pieces by stamping. In this way, lemon-flavored, non-sugar candy pieces with a water content of 2% by weight were obtained. The resulting, polyphenol-containing, non-sugar candy had no graininess and had a mild taste with the typical bitterness and astringency of the polyphenol kept low.

### EXAMPLE 7

In a vacuum kettle, reduced maltose syrup was concentrated by boiling until its water content reached 5% by weight or less, so that dough to be kneaded was obtained. Simphenon 90S was dissolved in glycerin in an amount equal to the weight of it. The Sunphenon 90S solution was added in an amount of 1.5% by weight to the dough, and they were mixed. In this process, 0.1% by weight of a herb flavor was added. The resulting kneaded dough was formed into 4 g spherical pieces by stamping. In this way, herb-flavored, non-sugar candy pieces with a water content of 2% by weight were obtained. The resulting, polyphenol-containing, non-sugar candy had no graininess in the mouth and had a mild taste with the typical bitterness and astringency of the polyphenol kept low.

### COMPARATIVE EXAMPLE 1

In a vacuum kettle, reduced maltose syrup was concentrated by boiling until its water content reached 5% by weight or less, so that dough to be kneaded was obtained. Oligonol was dissolved in glycerin in an amount twice the weight of Oligonol. The Oligonol solution was added in an amount of 3.6% by weight to the dough, and they were mixed. In this process, 0.1% by weight of a herb flavor was added. Ihe resulting kneaded dough was formed into 4 g spherical pieces by stamping. In this way, herb-flavored, non-sugar candy pieces with a water content of 2% by weight were obtained. The resulting, polyphenol-containing, non-sugar candy had no graininess in the mouth, but was uncomfortable to eat because the typical bitterness and astringency of the polyphenol were not suppressed enough. The candy began to become sticky fast, perhaps because the glycerin content was relatively high.

### COMPARATIVE EXAMPLE 2

In a vacuum kettle, reduced maltose syrup was concentrated by boiling until its water content reached 5% by weight or less, so that dough to be kneaded was obtained. Oligonol was dissolved in glycerin m an amount twice the weight of Oligonol. The Oligonol solution was added in an amount of 0.2% by weight to the dough, and they were mixed. In this process, 0.1% by weight of a herb flavor was added. The resulting kneaded dough was formed into 4 g spherical pieces by stamping. In this way, herb-flavored, non-sugar candy pieces with a water content of 2% by weight were obtained. Although having no graininess in the mouth, the resulting non-sugar candy had almost no characteristic polyphenol taste and was not acceptable as a polyphenol-containing, non-sugar candy that can be expected to have a physiological effect.

### COMPARATIVE EXAMPLE 3

In a vacuum kettle, reduced maltose syrup was concentrated by boiling until its water content reached 5% by weight or less, so that dough to he kneaded was obtained. To the dough was added 0.75% by weight of an Oligonol powder, and they were mixed. In this process, 0.1% by weight of a herb flavor was added. The resulting kneaded dough was formed into 4 g spherical pieces by stamping. In this way, herb-flavored, non-sugar candy pieces with a water content of 2% by weight were obtained. The resulting, polyphenol-containing, non-sugar candy had severe graininess in the mouth and also had a very strong level of the typical bitterness and astringency of the polyphenol.

Table 7 shows the composition and resulting, physical properties of the non-sugar candy obtained in each of Examples 4 to 7 and Comparative Examples 1 to 3. The physical properties were evaluated by 10 monitors based on the following evaluation criteria.

[Polyphenol Impression]

⊙: The non-sugar candy has a characteristic polyphenol taste.

∘: The non-sugar candy has a slight, characteristic polyphenol taste.

×: The non-sugar candy has no characteristic polyphenol taste.

The term “polyphenol impression” means that the typical bitterness and astringency of polyphenol is kept at a comfortable level so that the effect of the intake can be felt by feeling the presence of polyphenol.

[Masking of Bitterness and Astringency]

⊙: The non-sugar candy is easy to eat although it has bitterness and astringency.

∘: The non-sugar candy has slightly strong bitterness and astringency but is easy to eat.

×: The non-sugar candy has strong bitterness and stringency and is difficult to eat.

[Graininess]

⊙: The non-sugar candy has no graininess when licked.

×: The non-sugar candy has graininess when licked.

[Long-Term Stability]

⊙: The surface of the non-sugar candy takes a very long time to start becoming sticky.

∘: The surface of the non-sugar candy takes a long time to start becoming sticky.

×: The surface of the non-sugar candy takes a relatively short time to start becoming sticky.

The long-term stability was evaluated as follows. The non-sugar candy was allowed to stand for 6 weeks in a room where the temperature and the humidity were adjusted to 37° C. and 85%, respectively. Subsequently, the state of the non-sugar candy was evaluated by touching with finger. This long-term stability test is an accelerated test, which corresponds to a stability test by storage under normal distribution conditions at room temperature for about 1 year.

TABLE 7

| | Example | | | | Comparative Example | | |
|---|---|---|---|---|---|---|---|
| | 1 | 2 | 3 | 4 | 1 | 2 | 3 |
| Polyphenol (wt %) | 0.75 | 1 | 0.2 | 0.75 | 1.2 | 0.1 | 0.75 |
| Glycerin (wt %) | 0.75 | 2 | 0.2 | 0.75 | 2.4 | 0.1 | — |
| Polyphenol impression | ⊙ | ⊙ | ⊙ | ◯ | ⊙ | X | ⊙ |
| Masking of bitterness | ⊙ | ◯ | ⊙ | ⊙ | X | ⊙ | X |
| and astringency | | | | | | | |
| Graininess | ⊙ | ⊙ | ⊙ | ⊙ | ⊙ | ⊙ | X |
| Stability | ⊙ | ◯ | ⊙ | ⊙ | X | ⊙ | ⊙ |

The results in Table 7 show that the non-sugar candy obtained in each of Examples 4 to 7 had a good polyphenol impression, a high bitterness and astringency-masking effect, no graininess in the mouth, and good long-term stability as compared with that obtained in each of Comparative Examples 1 to 3.

### EXAMPLE 8

Four non-sugar candy products were each prepared as in Example 4, except that four polyphenols: Tannic Acid AL, Oseran, Oolong Tea Extract, and Blackberry Leaf Tea Extract were each used as the polyphenol soluble in glycerin.

The four non-sugar candy products had a good polyphenol impression, a high bitterness and astringency-masking effect, no graininess in the mouth, and good long-term stability similarly to that of Example 4.

### EXAMPLE 9

Seven non-sugar candy products were each prepared as in Example 4, except that propylene glycol was used as the water-soluble solvent and that seven polyphenols: Oligonol, Tannic Acid AL, Oseran, Oolong Tea Extract, Blackberry Leaf Tea Extract, Simphenon 90S, and resveratrol were each used as the polyphenol soluble in propylene glycol.

Although having a characteristic propylene glycol taste, these seven non-sugar candy products all had a good polyphenol impression, a high bitterness and astringency-masking effect, no graininess in the mouth, and good long-term stability similarly to that of Example 4.

(Effects)

The amount of low molecular proanthocyanidin can be reduced in the oral composition or confection of each example according to the present invention described above. Therefore, an oral composition and a confectionery product with an improved taste can be provided. Conventionally, it has been considered that low molecular proanthocyanidin is difficult to use in oral compositions and confections because the use of low molecular proanthocyanidin alone provides strong astringency and significantly deteriorates taste. However, the production method of the present invention makes it possible to produce an oral composition or confection containing low molecular proanthoeyanidin enough to provide anti-Candida activity.

The composition of the present invention, which contains the three specific ingredients: cinnamon, low molecular proanthocyanidin, and menthol, produces the above advantageous effects even when containing no decanoic acid. It should be noted that if decanoic acid is added in combination with the specific ingredients, a further advantageous effect can be expected, and therefore, the addition of decanoic acid should not he excluded from the scope of the present invention.

Conventionally, there has been a problem in that low molecular proanthocyanidin such as Oligonol has strong astringency and can deteriorate taste and therefore is not suitable for use in oral compositions and confections. However, the present invention makes it possible to provide oral compositions or confections that are effective in improving oral hygiene and have reduced astringency even though containing low molecular proanthocyanidin.

### INDUSTRIAL APPLICABILITY

The present invention is applicable in the field of oral hygiene improvement. An agent for promoting the secretion of salivary adiponectin can also be provided, so that further progress can be expected for lifestyle-related diseases such as metabolic syndrome, dry mouth, and a variety of health care regions including oral care.

1. An oral composition comprising at least three ingredients: cinnamon; low molecular proanthocyanidin; and menthol.

2. The oral composition according to claim 1, further comprising: at least one ingredient selected from a middle chain fatty acid, a middle chain fatty acid ester, and citral,

3. The oral composition according to claim 1, further comprising: a thickener.

4. The oral composition according to claim 1, wherein the cinnamon is a composition containing cinnamaldehyde.

5. The oral composition according to claim 1, further comprising at least one ingredient selected from decanoic acid or ethyl decanoate as a middle chain fatty acid or a middle chain fatty acid ester.

6. The oral composition according to claim 1, wherein the low molecular proanthocyanidin comprises Oligonol (trademark).

7. The oral composition according to claim 1, which contains at least 0.28 mg or more of cinnamon, 0.5 mg or more of low molecular proanthocyanidin, and 0.2 mg or more of menthol per oral dosage unit.

8. The oral composition according to claim 2, which contains at least one selected from 0.008 mg or more of a middle chain fatty acid, 0.008 mg or more of a middle chain fatty acid ester, or 0.14 mg or more of citral per oral dosage unit.

9. The oral composition according to claim 7, which promotes secretion of salivary adiponectin when orally taken to an organism.

10. A confection comprising at least three ingredients: cinnamon; low molecular proanthocyanidin; and menthol.

11. The confection according to claim 10, further comprising: at least one ingredient selected from a middle chain fatty acid, a middle chain fatty acid ester, and citral.

12. The confection according to claim 10, further comprising: a thickener.

13. The confection according to claim 10, wherein the cinnamon is a composition containing cinnamaldehyde.

14. The confection according to claim 10, further comprising at least one ingredient selected from decanoic acid or ethyl decanoate as a middle chain fatty acid or a middle chain fatty acid ester.

15. The confection according to claim 10, wherein the low molecular proanthocyanidin comprises Oligonol (trademark).

16. The confection according to claim 10, which contains at least 0.28 mg or more of cinnamon, 0.5 mg or more of low molecular proanthocyanidin, and 0.2 mg or more of menthol per orally dissolving unit.

17. The confection according to claim 11, which contains at least one selected from 0.008 mg or more of a middle chain fatty acid, 0.008 mg or more of a middle chain fatty acid ester, or 0.14 mg or more of citral per orally dissolving unit.

18. The confection according to claim 16, which is in the form of a candy product or a gel product and exhibits salivary adiponectin secretion activity when dissolved in a mouth of an organism.

19. (canceled)
